(19) United States
(12) Patent Application Publication
(10) Pub. No.: US 2008/0220525 A1
(43) Pub. Date: Sep. 11, 2008

(54) CULTURE SUBSTRATE PROVIDED WITH AN OXIDISED SILICONE COATING

(75) Inventors: Mathias BORELLA, Grenoble (FR); Marc Plissonnier, Eybens (FR); Emmanuelle Rouviere, Saint Egreve (FR); Frederic Gaillard, Voiron (FR)

Correspondence Address: OBLON, SPIVAK, MCCLELLAND MAIER & NEUSTADT, P.C., 1940 DUKE STREET, ALEXANDRIA, VA, 22314, US

(73) Assignee: COMMISSARIAT A L'ENERGIE ATOMIQUE, Paris (FR)

(21) Appl. No.: 11/839,777

(22) Filed: Aug. 16, 2007

(30) Foreign Application Priority Data
Sep. 7, 2006 (FR) 06 53607

(51) Int. Cl. C12N 5/06

(52) U.S. Cl. 435/402

(57) ABSTRACT

Solid culture substrate comprising, on at least one of its surfaces, a coating made of a material chosen from the oxidised silicones, said coating comprising a nanostructuration and/or a surface nanoroughness, and a surface nanoporosity.

Method for cultivating and growing living biomolecules in which the substrate is brought into contact with said living biomolecules and a growth culture medium.

### TECHNICAL FIELD

This invention relates to a culture substrate provided with an oxidised silicone coating.

More specifically, this invention relates to a culture substrate including, on at least one of its surfaces, a coating of a material chosen from the oxidised silicones.

The technical field of the invention can generally be defined as that of culture substrates, i.e. solid mechanical substrates designed to promote the fixation, attachment, growth and proliferation of “living biomolecules” such as those constituting living cells and tissues such as nerves.

For researches in biology, microbiology, medicine and pharmacology, the culture of living cells and tissues on mechanical media is very widely used.

In order for the experimental results of the analyses performed on these cells to be relevant and unambiguous, it is necessary to best adapt the culture environment so as to respect the physiological conditions necessary for the living biomolecules.

Regardless of the presence or lack thereof of a culture serum, the mechanical medium used for the culture is of utmost importance for a very large number of living cells and tissues as indicated in the document of Pool and Rappaport, Exp. Cell. Res., 20/465-510, 1960.

These substrates must offer perfect biocompatibility, be non-toxic, reproducible and allow for the adhesion and proliferation of cells so as to constitute a viable culture medium.

Thus, it is very common today in biology to use utensils of various shapes made for example of polystyrene (PS), polycarbonate (PC), polyethylene terephthalate (PET) or even silicate glass.

Polymer substrates have very good biocompatibility but very low surface energy. To improve their performance in terms of cell adhesion, it is very common to modify their surface by a plasma discharge treatment, for example.

However, the substrates described above are often very unsuitable for sensitive (i.e. difficult) cultures having more complex, more specific physiological needs, such as, for example, primary cells, hepatic cells, epithelial cells, neurons, bacteria, and so on.

The use of standard substrates with biomolecules that are difficult to cultivate makes the proliferation very slow, the expression of functions unremarkable and the differentiation particularly difficult.

To solve this specific technical problem, a number of solutions have already been proposed.

It was proposed, in document U.S. Pat. No. 4,243,692, to increase the specific contact surface of the medium, the mechanical culture substrate by coating a planar substrate with silicon heteropolycondensates in order to provide a better medium promoting the adhesion of the organisms and their development as a coherent group. This type of method is complex, difficult and relatively non-reproducible. In addition, it can leave toxic products from the condensation reaction on the substrate.

Again with the goal of increasing the specific contact surface and the number of anchoring points, it was proposed, in document US-A-2005 0095695, to produce cell cultures on polymer nanofibre stack-type structures. This method is effective but complex to implement and is in no way guaranteed to be effective for the culture of sensitive cells.

A very interesting solution was proposed in application WO-A-9712966, which consists of the liquid deposition of nanometric silica particles. This method has the advantage of being capable of covering a wide variety of shapes as well as allowing for deposition on very specific localised areas. However, in the method of this document, pure silica nanopowders are implemented, which is not always the best substrate for certain cells because it causes significant stress, and the addition of organic compounds can be done only be incorporating particles in the liquid phase. Finally, the most effective and reproducible substrates are obtained by “spin coating”, which significantly limits the shapes of substrates to be coated.

Moreover, none of the methods cited above make it possible to do without the addition of chemical substances to the culture medium so as to promote the adhesion and the expression of functions. While commonly used, these products have a detrimental action on biomolecules and cause significant stress and mortality, which does not always facilitate differentiation.

A substrate making it possible to avoid the use of these products would be an important advance for the culture of sensitive cells.

In regard to the above, there is a need for a culture substrate, for adhesion and fixation, the expression of living biomolecules in order in particular to produce a culture for cells and living tissues that is perfectly biocompatible, non-toxic, that allows for excellent adhesion, proliferation, differentiation of cells and tissues as well as an excellent expression of functions, without it being necessary to add promoter compounds promoting the adhesion, proliferation, differentiation and expression of functions to the culture medium.

There is also a need for such a culture substrate that can be produced in a large number by a simple, rapid, reliable, inexpensive and reproducible method with regard to the quality and the nature of the substrates obtained.

There is in particular a need for culture substrates that make it possible, while being easy to use and easy to produce in large numbers, to obtain excellent results in terms of adhesion, proliferation, expression of functions and differentiation in the culture of sensitive biomolecules, cells and tissues, i.e. which are difficult to cultivate, which require significant anchoring, of which the study requires the expression of specific functions and of which the differentiation is therefore difficult.

The goal of this invention is to provide a culture substrate that satisfies, among others, the needs listed above.

The goal of this invention is also to provide a culture substrate that does not have the drawbacks, defects, limitations and disadvantages of the substrates of the prior art, that solves the problems presented by the substrates of the prior art and that meets all the requirements and criteria for such a substrate.

This goal and others are achieved, according to the invention, by a solid culture substrate comprising, on at least one of its surfaces, a coating made of a material chosen from the oxidised silicones, said coating comprising a surface nanostructuration and/or nanoroughness, and a surface nanoporosity.

The cultures substrate according to the invention is defined by the combination of three essential features: the choice of an oxidised silicone for the material that constitutes the coating, the surface nanostructuration and/or nanoroughness of the coating, and finally the surface nanoporosity of the coating.

Such a culture substrate is neither described nor suggested in the prior art documents.

The culture substrate according to the invention does not have the inconveniences, defects, limitations and disadvantages of the substrates of the prior art. It satisfies the needs listed above, it satisfies the criteria and requirements for this type of substrate, and finally, it provides a solution to the problems presented by the culture substrates of the prior art.

The reunion, combination or association of the three features cited above, i.e. the surface nanostructuration and/or nanoroughness, surface nanoporosity, and the conformation of the oxidised silicone (these features, parameters preferably defining a specific domain) make it possible, in a surprising manner, to obtain excellent performance in terms of the cell culture, in particular with regard to the adhesion, the proliferation, the differentiation and the expression of functions, even without the addition of promoters or chemical substances promoting, for example, adhesion and expression, to the culture medium, such as dimethyl sulfoxide (DMSO).

The culture substrate of the invention is in particular especially suitable for delicate “sensitive” cells, of which the culture is difficult, which require significant anchoring, of which the study requires the expression of specific functions and of which the differentiation was difficult on the culture substrates of the prior art.

The substrate according to the invention defined by the association of the three features cited above is perfectly suitable for the culture of living biomolecules in general, and provides excellent results in the context of this culture, but also, surprisingly, also provides excellent results and performances for the culture of primary cells, hepatic cells, neurons and all sensitive and delicate cells. Thus, it was possible to observe in all of the cases a rapid proliferation, and the expression of functions, preferably without the incorporation of promoters, with a very high differentiation.

The invention will now be described in detail in the following description, provided for illustrative and non-limiting purposes in reference to the appended drawings, in which:

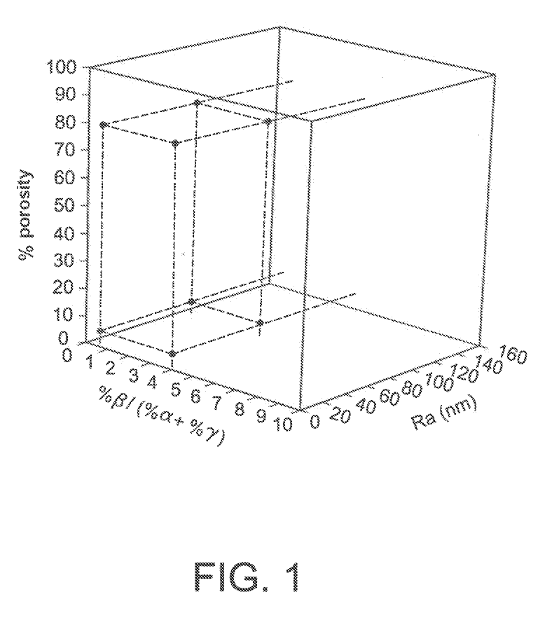

FIG. 1 is a three-dimensional graph showing the percentage of porosity (Z axis), the roughness Ra (nm), and the % β/(% α+% β) and showing the preferred range of definition of the coating of the substrate according to the invention;

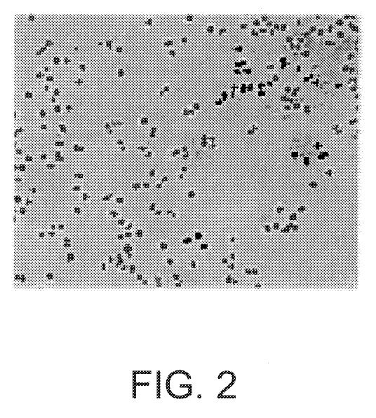

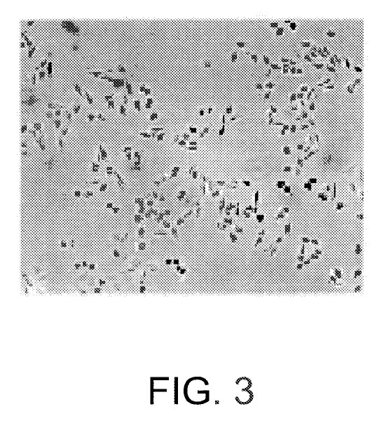

FIGS. 2 and 3 are microphotographs taken after two days of CHO cell culture respectively on substrate 1 and substrate 2 defined in example 2 below.

The material of the coating of the substrate according to the invention is an oxidised silicone generally comprising groups chosen from the groups SiO₂ (C, H)₂ (α), SiO₃ (C, H)(β) and SiO₄ (γ) .

According to the invention, at least one of these three groups is present, and preferably all three groups are present.

However, the material of the coating of the substrate according to the invention can be constituted not exclusively by the 3 groups α, β and γ above. It can further comprise one or more other groups different from groups α, β and γ so as to provide a total of 100%, such as groups (ω) SiO(C,H)₃.

The notation used above is clear to a person skilled in the art in this field and is commonly used. It is also possible to describe the (a) groups as SiO₂(R₁)(R₂) groups, the (β) groups as SiO₃(R₁) groups, the (γ) groups as SiO₄ groups and the possible (ω) groups as SiO(R₁)(R₂)(R₃) groups where R₁, R₂ and R₃, identical or different, are hydrocarbon groups such as linear or branched alkyl (1 to 10C) groups such as methyl, ethyl, n-propyl, i-propyl and n-butyl groups.

The proportions of the various groups (α), (β) and (γ) are adjusted in particular according to the needs of the application and of the culture to be produced, i.e. according to the biomolecule to be cultivated.

The following proportions are preferably used: 15 to 35% (α) groups, 30 to 80% (β) groups and 5 to 50% (γ) groups. In other words, the oxidized material preferably comprises 15 to 35% (α) groups, 30 to 80% (β) groups and 5 to 50% (γ) groups, more preferably the oxidized material consists of 15 to 35% (a) groups, 30 to 80% (β) groups and 5 to 50% (γ) groups. It would appear that with said preferred proportions the “stress” of the cultivated biomolecules is reduced.

In addition, the coating of the substrate according to the invention can be provided with additional functions (i.e. functions not initially belonging to the oxidised silicone) chosen according to the culture to be produced, to the type of biomolecules to be cultivated. These additional functions satisfy the specific physiological needs of each biomolecule and make it possible to further enhance the performance of the culture substrate according to the invention and to adapt it precisely to the culture to be produced.

These additional functions can generally be chosen from the hydroxyl groups (OH), the carboxyl groups (COOH), the methyl groups, and the bioactive elements such as Na and Ca.

The term “bioactive element” is a term known to the man skilled in the art and is commonly used in this technical field.

This term refers to an element, for example a particle or a molecule, that exerts an effect on a biomolecule, for example of a cell, of a tissue or of a nerve, etc.

The effects of the bioactive element can be of very different types: adhesion, non-adhesion, growth, nutrition, differentiation, therapeutic, electric, antioxidant, and so on.

Na and Ca are examples among hundreds of elements capable of having a bioactive nature. They have been chosen for their facility of integration within the actual coating of the invention during its development.

The second essential feature of the culture substrate according to the invention is the fact that the coating with which it is provided includes a surface nanostructuration and/or nanoroughness. This surface nanostructuration and/or nanoroughness can be obtained either by a preliminary structuration of the basic substrate, or more advantageously by the method of synthesis of the coating material.

By “and/or” in the terms “nanostructuration and/or nanoroughness”, we mean that the coating may have only a geometric nanostructuration, or only a nanoroughness, or it may have a nanoroughness on a geometric nanostructuration.

The third essential feature of the substrate according to the invention is the fact that the coating with which it is provided has a surface nanoporosity that can be obtained by the method of synthesis of the coating or by post-treatment, involving, for example, the use of a pore-forming material in the synthesis, which is subsequently removed with the generation of pores.

The nanostructuration and/or surface roughness (this concerns the roughness, including the roughness on a structuration) is characterised by a roughness Rₐ generally between 2 nm and 80 nm.

Rₐ is the mean roughness according to the standard DIN 4768.

It is defined by the arithmetic mean of all of the roughness values R on the evaluation length 1 (i.e. amplitude of the mean profile).

$${R}_{a} = \frac{1}{l} {\int }_{0}^{1} \vert z \vert \cdot d x$$

By nanostructuration, we generally mean that the coating comprises reliefs (humps) and/or recesses (hollows) i.e. raised and/or lowered reliefs.

These reliefs and/or recesses can in particular be in one or more of the following forms: trenches, grooves, slots, ridges, flutes, holes, cavities, pads, nibs, spikes, points, protuberances, asperities, bosses, projections, embossments, corrugations, valleys, peaks and channels.

This nanostructuration can in particular be in the form of peaks and valleys with a height h of the peaks generally between 4 and 160 nm, a width 1 of the peaks between 2 and 200 nm e.g. between 20 and 200 nm, and a ratio of peak width/valley width ranging from 0.2 to 2.

The surface nanoporosity of the coating material is generally defined by a porosity greater than or equal to 5%, preferably 5 to 80%. In general, the porosity is adapted to the biomolecule to be cultivated.

In addition, this nanoporosity preferably includes open pores with a size (diameter) generally not exceeding 25 nm in a proportion of 5 to 80% of the porous volume of the coating.

It should be noted that the coating is generally not entirely porous in its entire width and that only a more or less thick superficial layer is concerned by the pores.

An especially preferred material with particularly high performance for the cell culture in particular with regard to the adhesion, proliferation, differentiation, and expression of functions, is defined by specific ranges of porosity (in %), Rₐ (nm), and the ratio % β/(% α+% γ), which are as follows:
porosity: from 5 to 80%
Rₐ (nm): from 2 to 80 nm
Ratio % β/(% α+% γ): from 0.5 to 4.

The 3 groups α, β and γ do not always add up to 100%, and it is therefore entirely possible to obtain the ratio mentioned above.

The material defined by the specific ranges of each of these three parameters can be represented by the domain shown in FIG. 1. The thickness of said coating is generally from 10 to 100 nm, preferably 20 to 50 nm, and more preferably 30 to 40 nm. Thicknesses of 10, 15, or 25 nm can be used.

A low material thickness as defined above is sufficient, but it is nevertheless entirely possible to use greater thicknesses, namely for example a micron, or a plurality of microns.

The advantage of a low thickness lies in the fact that the material remains very largely transparent in the visible and has a very low induced fluorescence thus facilitating the optical analyses.

The substrate (medium, basic substrate to be distinguished from the culture substrate, which includes the medium, basic substrate and the coating) can be made of any other suitable solid material.

It can be any solid material known to a person skilled in the art such as, for example, the support materials used to produce analysis Microsystems and biochips. The material of the substrate can be organic or inorganic. It can be made of a material chosen from glass, silica, polycarbonates, polymethyl methacrylates (PMMA), polystyrenes, polyethylene terephthalates; the substrate can also be a composite substrate including a plurality of different materials chosen, for example, from those listed above.

The substrate, which is entirely or partially covered by the coating as described above can have any shape. It can in particular be any instrument, utensil or device traditionally used by a person skilled in the art for the culture of living biological molecules, cells and tissues.

The substrate according to the invention can be prepared according to a first embodiment by a method in which, a coating is deposited by PECVD in a single step on a substrate, from an organosilicon precursor mixed with oxygen, said coating being made of a material chosen from the oxidised silicones as described above.

In this embodiment of the method, the three features of the material according to the invention are obtained simultaneously.

The substrate has a nanostructuration and/or a nanoroughness that is found again in the deposited layer.

The precursor is chosen from the organosilicon compounds such as hexamethyldisiloxane, octamethylcyclotetra-siloxane, or decamethyltetrasiloxane.

A person skilled in the art will adjust the proportions of the mixture of precursor and oxygen, and the other parameters of the discharge such as the dissipated power, the working pressure, and the flow rates, so as to obtain an oxidised silicone having the desired composition and the desired proportions of groups (α), (β) and (γ).

The working pressure is an important parameter for obtaining the desired porosity.

The substrate according to the invention can be prepared according to a second embodiment by a method in which the following series of steps are performed:
a very poorly (weakly) crosslinked (reticulated) and very organic coating is deposited by PECVD from an organosilicon precursor mixed with a neutral gas,
the coating thus obtained is treated with a neutral or reductive gas plasma under conditions promoting the generation of gaseous species in the material so as to generate the nanoporosity and the nanoroughness.

The terms “very poorly (weakly) crosslinked” and “very organic” are known by the man skilled in the art and are very commonly used terms known in this field.

The terms “very crosslinked” (“high reticulation” or “high crosslinking”) or “poorly, weakly crosslinked” (“low reticulation” or “low crosslinking”) have a well-known meaning and mean that the lower, the crosslinking, reticulation of the material, the higher the number of chemical groups present in the precursor.

The term “very organic” means that the groups having carbons are predominant, which corresponds, for example to a strong presence of α and/or ω groups.

The precursor used in the first step is chosen according to its capacity to enable the solid coating material to form pores and the roughness a posteriori in the second step. This means that the precursor molecule must make it possible to form a solid deposit while maximising preservation of its organic part.

This precursor can be chosen from the organosilicons such as octamethylcyclotetrasiloxane, hexamethylcyclotrisiloxane and decamethyltetrasiloxane.

The precursor is diluted in a neutral gas such as argon or helium.

The deposition conditions in the first step are such that they enable the deposition of a low crosslinking, reticulation and very organic coating so as to enable the treatment of the second step to be effective. In other words, it is necessary to preserve the organic part constituted for example by the hydrocarbon groups, for example alkyl or methyl, of the precursors.

The coating obtained at the end of the first step can thus generally comprise a minimum of 60% (α) groups, around 20% (ω) groups, a maximum of 20% (β) groups and 0% (γ) groups.

According to the type of coating obtained at the end of the first step, the treatment of the second step will be adjusted. This treatment consists in promoting the formation of gaseous species within the coating material from its organic constituents, i.e. from hydrocarbon groups such as the alkyl groups such as (—CH₃) without oxidation thereof, because otherwise the formation of porosity and roughness does not occur.

It is therefore preferable to use a neutral gas, such as argon or helium, plasma, or a reductive gas such as hydrogen, plasma, with a dissipated power and a treatment time adapted to the initial material, for example, respectively several tens W/cm² or so and on the order of one minute and under a pressure, for example, between 10 and 10 Pa.

By adjusting the parameters of the plasma treatment of the second step, we obtain a coating having the desired composition defined by the proportions of (α), (β) and (γ) groups, the surface nanoporosity and the surface nanoroughnesses desired. Preferably, it is of course done so that the composition of the coating, the roughness and the porosity are in the preferred range indicated above and shown in FIG. 1.

The composition of the coating, namely the proportions of the various groups α, β, γ, the surface nanoporosity and the surface nanoroughness can be adjusted according to the biological molecules, cells or tissues to be cultivated.

The invention also relates to a method for cultivating, growing, living biomolecules in which the substrate as described above is brought into contact with said biomolecules and a growth culture medium.

The invention will now be described in reference to the following examples, provided for illustrative and non-limiting purposes.

### EXAMPLES

### Example 1

This example describes the production of a culture substrate according to the invention, particularly suitable for the physiological needs of a hepatocyte cell line hepaRG.

The substrate used is a standard optical microscopy glass plate that has a surface roughness Rₐ of 9 nm.

In a second step, a low crosslinking (reticulation) silicone-type material is deposited on this substrate by plasma-enhanced chemical vapour deposition (PECVD).

The precursor used in this example is decamethyltetrasiloxane (C₁₀H₃₀O₃Si₄).

This precursor is diluted in helium in a 20% proportion.

The deposition conditions are as follows: 0.2 W/cm², 25 Pa, 75 seconds.

At the end of this first step, a coating with a thickness of 100 nm on the glass substrate is obtained.

The material deposited includes a minimum of 60% (α) groups, around 20% SiO(C,H)₃ groups, a maximum of 20% (β) groups and 0% (γ) groups.

The solid material thus deposited has a very low reticulation and very organic conformation, i.e. this conformation is very similar to that of the precursor molecule with the preservation of the organic groups of the latter. The terms “(very low) reticulation” and “very organic” were defined in detail above.

In a second step, the treatment of the coating deposited in the first step is carried out using a helium plasma with a dissipated pressure of 0.5 W/cm² and a treatment time of 60 seconds under a pressure of 50 Pa. Such conditions promote the formation of gaseous species such as CH₄, C₂H₂, C₂H₄ within the material from its organic constituents (essentially CH₃ groups) without oxidation thereof. This results in the formation of porosity and roughness of the surface of the material deposited in the first step.

The plasma treatment of the second step results in a coating made of a material comprising 25% (α) groups, 65% (β) groups and 10% (γ) groups, +/−10%.

This treatment also made it possible to obtain a coating comprising a roughness Rₐ of around 3 nm, which is superimposed on the roughness of the glass substrate, and a surface porosity of around 10%, on 10 to 15 nm of thickness.

The roughness Rₐ of the substrate was determined by atomic force microscopy and the porosity was measured by spectroscopic ellipsometry.

This substrate is particularly suitable for the cell of a hepatocyte cell line hepa RG.

With the substrate according to the invention prepared in this example, the generation time of the hepatocytes, i.e. the time necessary for the number of cells to double, was divided by two with respect to a standard polystyrene culture substrate.

In addition, the cells show a minimum stress lower than all of the culture substrates of the prior art such as the culture glass and polystyrene used for the purpose of comparison.

Finally, the differentiation of the cells is exceptional, and without the addition of a promoter.

The stress and the differentiation (which is the individual behaviour with respect to the behaviour of the group) are criteria and notions that are well known in biology and are generally qualitatively assessed.

### Example 2

In this example, we compare cultures produced on two different substrates of which one (substrate 2) is consistent with the invention.

Substrate 1 is characterised by the presence of an oxidised silicone including 10% of (α), 25% of (β) and 65% of (γ), a roughness Ra of 10 nm and zero porosity.

Substrate 2 is characterised by the presence of an oxidised silicone including 30% of (α), 55% of (β) and 15% of (γ), a roughness Ra of 18 nm and a surface porosity of around 20% on 20 to 25 nm of thickness.

More specifically, in this example, we compare the growth of CHO (Chinese Hamster Ovary) cells on these two substrates. The procedure is as follows.

A CHO (Chinese Hamster Ovarian Cell) cell line, adherent cells, was cultivated in a Petri dish until confluency. The cells were collected by trypsinization as follows.

The culture medium is removed. The culture is cleaned by two washings of 2-3 ml of PBS. Then, around 1 ml of trypsin is added per 50 cm² of surface to be trypsinized. The culture is placed at 37° C. for 1 minute. The trypsin is delicately removed by pipetting. The dish is returned to 37° C. for 3 minutes. The cells are collected in the culture medium (DMEM+antibiotic) by agitation and flushing. They are re-suspended in a volume at least 3 times larger, then the entirety is vortexed.

Plates forming substrate 1 or substrate 2 were placed by 3 in square culture dishes with a surface of 120 cm². Each dish was seeded with 50 ml of culture suspension in an amount of 6.6×10⁴ cells/ml (i.e. a distribution in principle of 55 cells/cm² in the dish).

The two substrates 1 and 2 make it possible to obtain, for the CHO cells, a comparable generation time on the order of 20 h.

The generation time is defined by

$$Tg = \frac{( T - {T}_{0} ) \times ln 2}{ln N - ln {N}_{0}} ,$$

Tg is the generation time. T−T₀ is the counting time. N is the number of cells at T. N₀ is the number of cells at T₀.

However, the size of the spread of cells after 2 days of culture attests to the minimal stress of the substrate 2 according to the invention and its better suitability for the physiological needs of cells (see FIGS. 2 and 3).

1. Solid culture substrate comprising, on at least one of its surfaces, a coating made of a material chosen from the oxidised silicones, said coating comprising a nanostructuration and/or a surface nanoroughness, and a surface nanoporosity.

2. Substrate according to claim 1, wherein the nanostructuration and/or nanoroughness is characterised by a roughness Rₐ according to standard DIN 4768 between 2 nm and 80 nm.

3. Substrate according to any one of the preceeding claims, wherein the surface nanostructuration comprises reliefs and/or recesses.

4. Substrate according to claim 3, wherein the surface nanostructuration of the coating is in one or more of the following forms: trenches, grooves, slots, ridges, flutes, holes, cavities, pads, nibs, spikes, points, protuberances, asperities, bosses, projections, embossments, corrugations, valleys, peaks and channels.

5. Substrate according to claim 4, wherein the surface nanostructuration and/or nanoroughness of the coating is in the form of peaks and valleys with a height h of the peaks between 4 and 160 nm, a width 1 of the peaks between 2 and 200 nm, and a ratio of peak width/valley width ranging from 0.2 to 2.

6. Substrate according to any one of the preceeding claims, wherein the nanoporosity of the material is greater than or equal to 5%, preferably 5 to 80%.

7. Substrate according to any one of the preceeding claims, wherein the nanoporosity includes open pores with a size not exceeding 25 nm in a proportion of 5 to 80% of the porous volume of the coating.

8. Substrate according to any one of the preceeding claims, wherein the material of the coating comprises groups chosen from the groups SiO₂(C,H)₂(α), SiO₃(C,H)(β) and SiO₄(γ).

9. Substrate according to claim 8, wherein the material of the coating further comprises one or more other groups different from the (α), (β) and (γ) groups such as SiO(C,H)₃(ω) groups.

10. Substrate according to claim 8, wherein the material of the coating comprises, preferably consists of, 15 to 35% (α) groups, 30 to 80% (β) groups and 5 to 50% (γ) groups.

11. Substrate according to any one of the preceeding claims, wherein the coating of the substrate is further provided with additional functions chosen from the hydroxyl groups, the carboxyl groups, the methyl groups, and the bioactive elements such as Na and Ca.

12. Substrate according to any one of the preceeding claims, wherein the thickness of said coating is 10 to 100 nm, preferably 20 to 50 nm, and more preferably 30 to 40 nm.

13. Method for preparing the culture substrate according to any one of claims 1 to 12, wherein a coating made of a material chosen from the oxidised silicones, is deposited on a substrate by PECVD, in a single step, from an organosilicon precursor mixed with oxygen.

14. Method according to claim 13, wherein said organosilicon precursor is chosen from hexamethyldisiloxane, octamethylcyclotetrasiloxane, and decamethyltetrasiloxane.

15. Method for preparing the culture substrate according to any one of claims 1 to 12, wherein the following successive of steps are performed:
a very poorly crosslinked and very organic coating is deposited by PECVD from an organosilicon precursor mixed with a neutral gas,
the coating thus obtained is treated with a neutral or reductive gas plasma under conditions promoting the generation of gaseous species in the material so as to generate the nanoporosity and the nanoroughness.

16. Method for cultivating, growing living biomolecules in which the substrate according to any one of claims 1 to 12 is brought into contact with said living biomolecules and a growth culture medium.
